(19) United States
(12) Patent Application Publication
(10) Pub. No.: US 2006/0188389 A1
(43) Pub. Date: Aug. 24, 2006

(54) Method and system for reducing microbes on a portable electronic device

(75) Inventor: Rami C. Levy, Plantation, FL (US)

Correspondence Address: AKERMAN SENTERFITT, P.O. BOX 3188, WEST PALM BEACH, FL, 33402-3188, US

(73) Assignee: Motorola, Inc., Schaumburg, IL (US)

(21) Appl. No.: 11/068,651

(22) Filed: Feb. 24, 2005

(51) Int. Cl. A61L 2/00; B01J 19/12

(52) U.S. Cl. 422/24; 422/186.3

(57) ABSTRACT

A system (11) for reducing microbes such as bacteria or germs on a portable electronic device (10) can include a portable communication device having a sensor (32) for detecting a condition of the portable communication device and a light source (30) selectively activated for germicidal reduction based on the condition. The condition can include a charging status for the portable communication device or a position for a flip on the portable electronic device, or any number of other operating or environmental conditions. The light source can be an ultra-violet light source such as a UV-A, UV-B, or UV-C light emitting diode. The system can optionally include a charging base (34) having a light source (36) such that the source operates as a anti-bacterial light source when the portable electronic device is being charged.

### FIELD OF THE INVENTION

This invention relates generally to reducing microbes, and more particularly to a method and system for reducing microbes on portable electronic devices.

### BACKGROUND OF THE INVENTION

Microphones (on cell phones or desk phones, for example) are breeding grounds for microorganisms such as germs and bacteria. There are several passive solutions that attempt to sanitize the microphone area in some way using anti-microbial films or other anti-bacterial chemicals that can be embedded into the housings of such products. Other areas of the phone can also become contaminated due to frequent handling (touchscreen, phone body and keypad for example) and might not be easily sanitized using such existing techniques. There are also toothbrush sterilization systems that use germicidal UV lights.

### SUMMARY OF THE INVENTION

Embodiments in accordance with the present invention provide a system and method for sensing a condition on a portable electronic device such as a portable communication device and reducing germs or microbes selectively using a light source based on the condition sensed at the portable electronic device.

In a first embodiment of the present invention, a system for reducing microbes such as bacteria or germs on a portable electronic device can include a portable communication device having a sensor for detecting a condition of the portable communication device and a light source selectively activated for germicidal reduction based on the condition. The condition can include a charging status for the portable communication device or a position for a flip on the portable electronic device, or any number of other operating or environmental conditions. For example, the system can include an environmental sensor for detecting a proximity (or lack thereof) to a human. The light source can be an ultra-violet light source such as a UV-A, UV-B, or UV-C light emitting diode or an electroluminescent (EL) light device. The system can further optionally include a charging base or other receptacle having a light source such that the light source operates as an anti-bacterial light source when the portable electronic device is being charged or received by the receptacle. Note, the portable electronic device can be any number of devices such as a cellular phone, a home cordless phone, a smart phone, a personal digital assistant, a wireless messaging device, an MP3 player, a shaver, a toothbrush, or a personal hygiene device.

In a second embodiment of the present invention, a system for reducing microbes on at least portions of a portable communication product can include a sensor for detecting at least one condition among an operational condition and an environmental condition of the portable communication product and an ultra-violet light source selectively activated for germicidal sterilization based on the condition detected. The sensor can detect at least one condition selected among a charging status for the portable communication device, a position for a flip on the portable communication product, a proximity or distance to a human, and a talk condition sol the portable communication device. A talk condition can be a condition when a user is typically speaking through a microphone and listening through a earpiece on the portable communication product.

In a third embodiment of the present invention, a method of reducing microbes on a portable electronic device can include the steps of sensing for a predetermined condition on the portable communication device and selectively activating a light source for microbial/germicidal sterilization based on the predetermined condition sensed. The light source can be an ultra-violet light source such as a UV-A, UV-B, or UV-C light emitting diode or other light sources. The step of sensing the predetermined condition can include sensing a charging status for the portable communication device, or sensing a closed position for a flip on the portable electronic device, or sensing using an environmental sensor for a predetermined proximity to a human. The method can further include the step of deactivating the light source when detecting the predetermined proximity to the human. In one alternative, the step of selectively activating the ultra-violet light source can include activating the ultra-violet light source as a anti-bacterial light source when the portable electronic device is being charged in a charging base.

Other embodiments, when configured in accordance with the inventive arrangements disclosed herein, can include a system for performing and a machine readable storage for causing a machine to perform the various processes and methods disclosed herein.

### BRIEF DESCRIPTION OF THE DRAWINGS

FIG. 1 is a block diagram of a system for reducing microbes on a portable electronic device in accordance with an embodiment of the present invention.

FIG. 2 is an illustration of a portable communication product having a system for reducing microbes in accordance with an embodiment of the present invention.

FIG. 3 is another illustration of a portable electronic device and a charging system having a system for reducing microbes in accordance with an embodiment of the present invention.

FIG. 4 is a flow chart illustrating a method of reducing microbes on portable electronic devices in accordance with an embodiment of the present invention.

### DETAILED DESCRIPTION OF THE DRAWINGS

While the specification concludes with claims defining the features of embodiments of the invention that are regarded as novel, it is believed that the invention will be better understood from a consideration of the following description in conjunction with the figures, in which like reference numerals are carried forward.

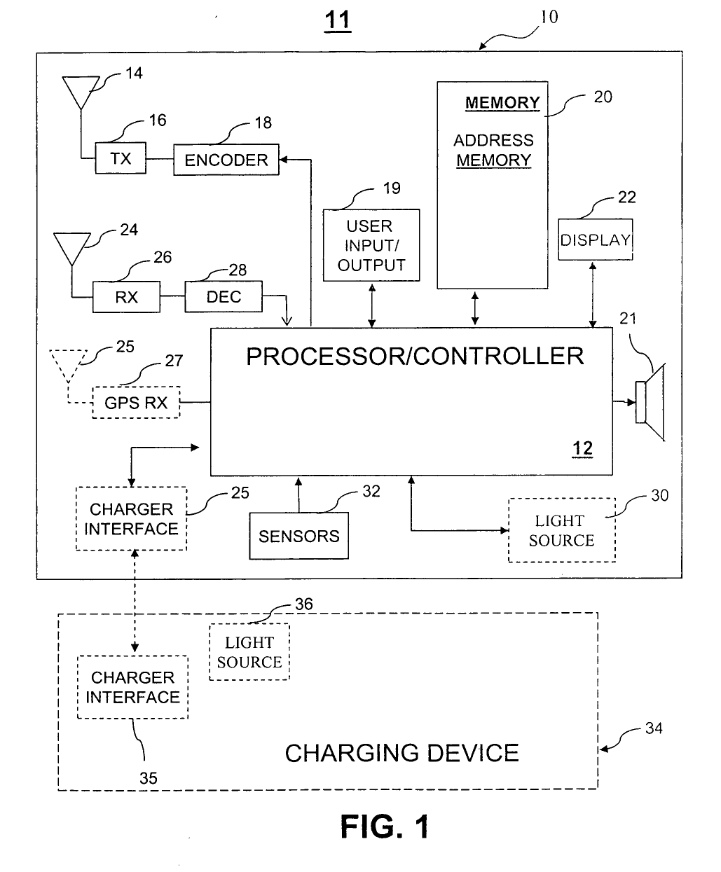

Referring to FIG. 1, a system 11 having a portable electronic device 10 such as a mobile telephone or camera phone can include a processor 12 programmed to function in accordance with the described embodiments of the present invention. The portable electronic device 10 can essentially be any electronic device having sensors for detecting predetermined conditions in the portable electronic device 10. Examples of devices within contemplation of the various embodiments herein can include, but are not limited to cell-phones, home cordless phones, smart-phones, PDAs, laptop computers, pocket PCs, DVD players, personal audio/video playback devices such as CD & MP3 players, remote controllers, electronic gaming devices, shavers, toothbrushes, and any other personal hygiene device.

The portable electronic device 10 can optionally include (particularly in the case of a cell phone or other wireless device) an encoder 18, transmitter 16 and antenna 14 for encoding and transmitting information as well as an antenna 24, receiver 26 and decoder 28 for receiving and decoding information sent to the portable electronic device 10. The portable electronic device 10 can further include a memory 20, a display 22 for displaying a graphical user interface or other presentation data, and a speaker 21 for providing an audio output. The processor or controller 12 can be further coupled to the display 22, the speaker 21, the encoder 1 8, the decoder 28, and the memory 20. The memory 20 can include address memory, message memory, and memory for database information.

Additionally, the portable electronic device 10 can include user input/output device(s) 19 coupled to the processor 12. The input/output device 19 can be a microphone for receiving voice input or a low audio speaker used as an earpiece for providing an audio output. Of course, input/output device 19 can also be a keyboard, a keypad, a handwriting recognition tablet, or some other Graphical User Interface for entering text or other data. Optionally, the portable electronic device 10 can further include a GPS receiver 27 and antenna 25 coupled to the processor 12 to enable location determination of the communication device. Of course, location or estimated location information can be determined with just the receiver 26 using triangulation techniques or identifiers transmitted over the air.

Further note, the system 11 can optionally include a charger 34 having an interface 35 that couples the portable electronic device 10 via the charging interface 25 of the device 10. Also note, the system can include any number of light sources 30 in the portable electronic device 10 or optionally light sources 36 in the charging device. The light sources can be ultra-violet lights such as UV-A, UV-B, or UV-C light emitting diodes that can reduce germs or microbes or otherwise serve as a germicide or sanitation device. Although, UV-C appears to be the most effective light source against germs, other light sources can be used to reduce germs or microbes either using the radiation or thermal characteristics of the light source to provide such function. Additionally note that the system 11 can alternatively include docking devices or holsters (instead of a charging base) that have a receptacle including such light sources for providing the function of reducing microbes or germs as contemplated herein. Note, in some embodiments, the system can be the charging device or a receptacle device itself having a sensor and light sources that are selectively activated based on an environmental or operational condition sensed or detected.

The portable electronic device 10 can also include any number of specific sensors 32 that can detect interaction with a user. Such sensors can include, but are not limited to heart rate sensors (e.g., ecg, pulse oximetry), blood oxygen level sensors (e.g., pulse oximetry), temperature sensors (e.g., thermocouple, IR non contact), eye movement and/or pupil dilation sensors, motion sensing (e.g., strain gauges, accelerometers, rotational rate meters), breathing rate sensors (e.g., resistance measurements, strain gauges), Galvanic skin response sensors, audio level sensing (e.g., microphone), force sensing (e.g., pressure sensors, load cells, strain gauges, piezoelectric). Each of these sensors can measure a condition such as a proximity to a user and/or an environmental condition that will assist the portable electronic device 10 to infer a proximity of the user to the portable electronic device. The sensor or sensors 32 can also simply be sensors that detect a particular state of a device. For example, a sensor in this regard can simply detect if a flip is closed on a flip phone or is being charged in a charging device 34 via charger interface 35. In most instances, the sensors can provide a means for enabling the sanitizing of the device when a user is not interacting with the device.

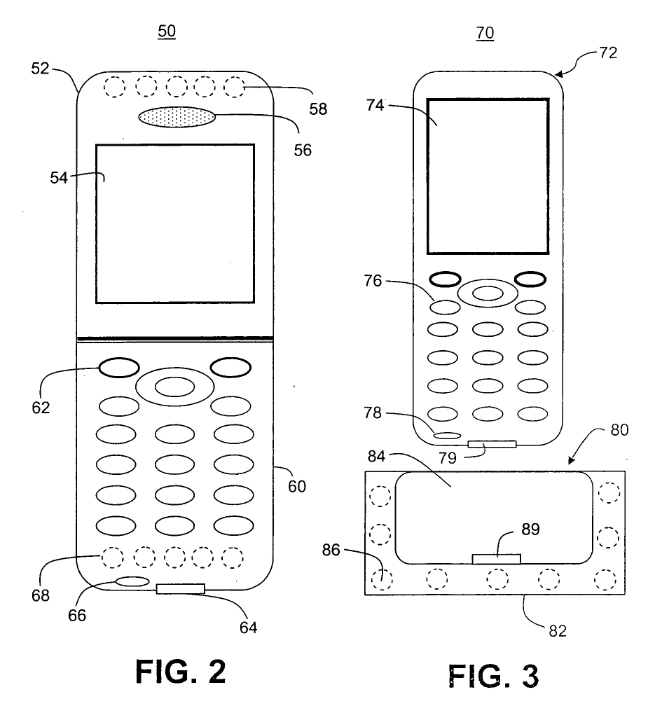

Referring to FIG. 2, a portable communication device 50 is shown in the form of a flip phone. The portable communication device 50 can include a flip portion 52 having a display 54, an earpiece speaker 56 and an optional set of lights 58 used for selective sanitation as described above. The device 50 further includes a main portion 60 having a set of keys 62, a microphone 66, a charger and/or programming interface 64, as well as another optional set of lights 68 used for selective sanitation or sterilization. As mentioned above, a sensor within the device can simply detect when the flip is closed and then selectively activate lights (58 or 68) strategically placed within the housing to reduce germs or microbes in locations most likely to inhabit the device 50. Note, the location of the lights are not necessarily limited to the areas shown, but can be placed anywhere either internally or externally to provide the function of reducing germs or microbes in accordance with the embodiments herein. Further note, the sensor (not shown in FIG. 2, but see item 32 in FIG. 1) can also be a camera on either the flip portion 52 or the main portion 60 that detects microbes or germs. In such an instance, any number of lights can be further selectively activated based on where the microbes or germs were detected.

Regarding FIG. 3, another system 70 is shown having a portable electronic device 72 such as a monolith shaped cellular phone or PDA and a corresponding receptacle device 80 such as a charging device, a docking port or a device holster. The device 72 can include a display 74, a plurality of keypads 76 and a charging and/or programming interface 79. In the case of a phone, the device 72 can further include a microphone 78. The device 72 can include lights (not shown) that can be selectively activated when a predetermined condition is detected. As noted before such conditions can include environmental or operational conditions that are indicative of a lack of user interaction or proximity to the device 72. In one embodiment, the device 72 can be placed in the receptacle device 80 having a housing 82 and a receptacle area 84 for receiving the device 72. The interface 79 of the device 72 can mate with an interface 89 of the device 80. Once the device 72 is detected as being within the receptacle area 84, one or more of a set of light sources 86 within the receptacle device 80 can be selectively activated. Note, the light sources 86 only need to be activated for a predetermined time and do not necessarily need to be on all the time. Thus, a processor within the receptacle device 80 or the device 72 can be used to limit the time such light sources remain on.

With respect to the light sources discussed above, it should be noted that the ultraviolet spectrum is broken up into three distinct bands, namely UV-A (400-315 nm) [a.k.a., Long Wave or “blacklight”], UV-B (315-280 nm) [a.k.a., Medium Wave], and UV-C (280-100 nm) [a.k.a., Short Wave or “germicidal”]. The general rule for disinfection is 16 milliwatt-seconds (mW*s) of UV optical energy per cm². Maximum germicidal effectiveness is at approximately 254 nm in the UV-C band. Typical toothbrush sterilizers disinfect 4 toothbrushes in 30 seconds to 6 minutes. Such toothbrush sterilizers use a 4 W UV-C lamp running on 6-9 VDC. Typical current is 300 mA. With this in mind, a smaller bulb (such as a UV-LED) should suffice for a smaller area around a phone microphone approximating 4 cm². As previously noted, the light sources (e.g., LEDs, EL panels, or otherwise) can be in the flip portion or near the microphones or speakerphone area, with their energy directed at the microphone or other area most likely to have microbial or germ infestation. The light source such as the LED can be activated periodically or when the phone is not in use (for example, once a day, or when the flip is closed).

If current drain proves to be an issue for the active LEDs in any particular embodiment, the UV LEDs could be alternatively mounted on a charger base (or other powered receptacle base) and the phone sterilized only when being charged or otherwise coupled to the base. Note, in one embodiment, the LEDs within the portable electronic device can be powered only by the charger when coupled to the charger. Of course, other embodiments can allow the LEDs within the portable electronic device to be activated using power from either the charger base or the portable electronic product or both. Although UV-A LEDs have a typical output power of 10 mW and an output efficiency of 10%, UV-C LEDs typically emit optimal power of about 1 mW, and have a typical 1-2% power output efficiency. To be effective, the LEDs can be on for about 3 minutes (assuming a 4 sq cm coverage area). (This is based on Sandia Labs' data with 275 nm LEDs, and assumes 20 mA input across a 3.7V drop (74 mW input power)).

Although there may be safety issues with respect to UV-C exposure, care can be taken such that the LEDs or other light sources are never active when the phone is open or in a condition likely to expose such light to a user. Such conditions can include when a flip on a flip phone is open or when the phone is near a heat source (human body, human face). Most UV-LEDs produced today have optimal intensity in the UV-A band, anywhere from 350 nm to 395 nm. However, in November 2003 Sandia Labs produced UV LEDs in the 290 nm (UV-B) and 275 nm (UV-C) range.

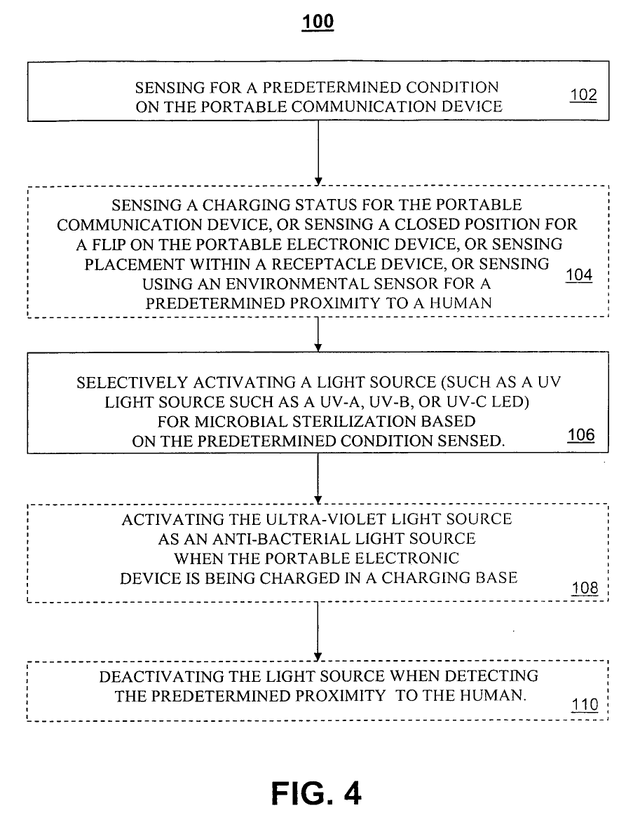

Referring to FIG. 4, a method 100 of reducing microbes on a portable electronic device can include the step 102 of sensing for a predetermined condition on the portable communication device and selectively activating a light source at step 106 for reducing microbes or germicidal sterilization based on the predetermined condition sensed. The light source can be an ultra-violet light source such as a UV-A, UV-B, or UV-C light emitting diode or other light source providing antibacterial or germicidal characteristics for example. Note, the step of sensing the predetermined condition can optionally include the step 104 of sensing a charging status for the portable communication device, or sensing a closed position for a flip on the portable electronic device, or sensing placement within a holster or other receptacle, or sensing by using an environmental sensor for a predetermined proximity or approximate distance to a human. In one alternative, the step of selectively activating the ultra-violet light source can optionally include the step 108 of activating the ultra-violet light source as a anti-bacterial light source when the portable electronic device is being charged in a charging base. The method 100 can further include the step 110 of deactivating the light source when detecting the presence of a human at or near a predetermined distance or proximity. Thus, embodiments herein actively reduce or eliminate bacteriological agents from a portable electronic device such as a communication device in an integrated form factor within a device or a charger that coupled to the device.

In light of the foregoing description, it should be recognized that embodiments in accordance with the present invention can be realized in hardware, software, or a combination of hardware and software. A network or system according to the present invention can be realized in a centralized fashion in one computer system or processor, or in a distributed fashion where different elements are spread across several interconnected computer systems or processors (such as a microprocessor and a DSP). Any kind of computer system, or other apparatus adapted for carrying out the functions described herein, is suited. A typical combination of hardware and software could be a general purpose computer system with a computer program that, when being loaded and executed, controls the computer system such that it carries out the functions described herein.

In light of the foregoing description, it should also be recognized that embodiments in accordance with the present invention can be realized in numerous configurations contemplated to be within the scope and spirit of the claims. Additionally, the description above is intended by way of example only and is not intended to limit the present invention in any way, except as set forth in the following claims.

What is claimed is:

1. A system for reducing microbes on a portable electronic device, comprising:
a portable communication device having a sensor for detecting a condition of the portable communication device; and
a light source selectively activated for microbial reduction based on the condition.

2. The system of claim 1, wherein the condition comprises a charging status for the portable communication device.

3. The system of claim 1, wherein the condition comprises a closed position for a flip on the portable electronic device.

4. The system of claim 1, wherein the sensor is an environmental sensor for detecting a lack of a predetermined proximity to a human.

5. The system of claim 1, wherein the condition comprises at least one among a lack of a talk condition, placement within a holster, and a docking within a docking station.

6. The system of claim 1, wherein the portable electronic device comprises at least one among a cellular phone, a home cordless phone, a smart phone, a personal digital assistant, a wireless messaging device, an MP3 player, a shaver, a toothbrush, and a personal hygiene device.

7. The system of claim 1, wherein the light source is an ultra-violet light source.

8. The system of claim 7, wherein the ultra-violet light source comprises at least one among a UV-A, UV-B, or UV-C light emitting diode.

9. The system of claim 1, wherein the system further comprises a charging base having the ultra-violet light source such that the ultra-violet light source operates as a anti-bacterial light source when the portable electronic device is being charged.

10. A system for reducing microbes on at least portions of a portable communication product, comprising:
a sensor for detecting at least one condition among an operational condition and an environmental condition of the portable communication product; and
an ultra-violet light source selectively activated for microbial sterilization based on the condition detected.

11. The system of claim 10, wherein the sensor detects at least one condition selected among a charging status for the portable communication device, a position for a flip on the portable communication product, a proximity to a human, and a talk condition for the portable communication device.

12. The system of claim 10, wherein the system comprises at least one among a cellular phone, a home cordless phone, a smart phone, a charging base, a docking station, and a wireless messaging device.

13. A method of reducing microbes on a portable electronic device, comprising the steps of:
sensing for a predetermined condition on the portable communication device; and
selectively activating a light source for microbial sterilization based on the predetermined condition sensed.

14. The method of claim 13, wherein the step of sensing the predetermined condition comprises sensing a charging status for the portable communication device.

15. The method of claim 13, wherein the step of sensing the predetermined condition comprises sensing a closed position for a flip on the portable electronic device.

16. The method of claim 13, wherein the step of sensing comprises the step of sensing using an environmental sensor for detecting a predetermined proximity to a human.

17. The method of claim 16, wherein the method further comprises the step of deactivating the light source when detecting the predetermined proximity to the human.

18. The method of claim 13, wherein the step of selectively activating the light source comprises selectively activating an ultra-violet light source.

19. The method of claim 18, wherein the step of selectively activating the ultra-violet light source comprises selectively activating at least one among a UV-A, UV-B, or UV-C light emitting diode.

20. The method of claim 18, wherein the step of selectively activating an ultra-violet light source comprises activating the ultra-violet light source as a anti-bacterial light source when the portable electronic device is being charged in a charging base.
